(19) United States
(12) Patent Application Publication
(10) Pub. No.: US 2013/0210911 A1
(43) Pub. Date: Aug. 15, 2013

(54) METHOD AND COMPOSITION FOR TREATING A DISEASE OR CONDITION RELATED TO OREXIN RECEPTOR 1, OREXIN RECEPTOR 2, SOMATOSTATIN RECEPTOR 2 OR DOPAMINE D2L RECEPTOR

(71) Applicants: Huai-Cheng Lee, Taipei City (TW); Guang-Tzuu Shane, Taipei City (TW); Hsi-Chieh Wang, Taipei CIty (TW); Rong Jin Lin, Taipei City (TW)

(72) Inventors: Huai-Cheng Lee, Taipei City (TW); Guang-Tzuu Shane, Taipei City (TW); Hsi-Chieh Wang, Taipei CIty (TW); Rong Jin Lin, Taipei City (TW)

(73) Assignee: CENTER LABORATORIES, INC., Taipei City (TW)

(21) Appl. No.: 13/509,221

(22) PCT Filed: Oct. 25, 2010
(86) PCT No.: PCT/CN10/01681

§ 371 Date: May 10, 2012

Related U.S. Application Data
(60) Provisional application No. 61/259,688, filed on Nov. 10, 2009.

(51) Int. Cl. A61K 31/277

(52) U.S. Cl. CPC A61K 31/277
USPC 514/523; 558/408; 435/375

(57) ABSTRACT

Disclosed herein are methods and composition for treating a disease or condition related to orexin receptor 1, orexin receptor 2, somatostatin receptor 2 or dopamine D_{2L} receptor. The method comprises administering a composition comprising an effective amount of verapamil and a pharmaceutically acceptable excipient to a subject, wherein the verapamil is capable of binding to at least one of a receptor selected from the group consisting of orexin receptor 1, orexin receptor 2, somatostatin receptor 2 and dopamine D_{2L} receptor of the subject.

### CROSS REFERENCE TO RELATED APPLICATIONS

This application is a U.S. National Stage of International Application No. PCT/CN2010/001681, filed on Oct. 25, 2010, for which priority is claimed under 35 U.S.C. §120; and this application claims priority of U.S. Provisional Application No. 61/259,688, filed on Nov. 10, 2009, under 35 U.S.C. §119(e), the entire contents of all of which are hereby incorporated by reference.

### TECHNICAL FIELD

This disclosure in general relates to the field of pharmacology.

### BACKGROUND ART

### DESCRIPTION OF RELATED ART

Objectives of this disclosure are identification of compounds that may bind to endogenous neurotransmitter receptors. Accordingly, verapamil and their stereoisomers, metabolites, derivatives, salts, solvates and combinations thereof are identified and formulated into a pharmaceutical composition for treating a disease or condition related to orexin receptor 1, orexin receptor 2, somatostatin receptor 2 or dopamine D_{2L} receptor.

### SUMMARY

As embodied and broadly described herein, disclosure herein features methods and composition for treating a disorder related to orexin receptor 1, orexin receptor 2, somatostatin receptor 2 or dopamine D_{2L} receptor.

Therefore, it is the first objective of this disclosure to provide a method of binding at least one of orexin receptor 1, orexin receptor 2, somatostatin receptor 2 or dopamine D_{2L} receptor in a subject, comprising the step of administering verapamil to the subject. In some embodiments, the verapamil is any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof. In one specific example, the verapamil is a racemic mixture of (R)-(+)-verapamil and (S)-(−)-verapamil. It is found that both (R)-(+)-verapamil hydrochloride and (S)-(−)-verapamil hydrochloride act as agonists to somatostatin receptor 2, and antagonists to orexin receptor 1, orexin receptor 2 or dopamine (D_{2L}) receptor.

It is the second objective of this disclosure to provide a method of treating a subject having a disease or condition related to orexin receptor 1, orexin receptor 2, somatostatin receptor 2 or dopamine D_{2L} receptor, comprising the step of administering an effective amount of verapamil and a pharmaceutically acceptable excipient to the subject, wherein the verapamil is any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof. In one preferred embodiment, the verapamil is (R)-(+)-verapamil, a pharmaceutically acceptable derivative, stereoisomer, metabolite, salt or solvate thereof, or a combination thereof.

In some embodiments, the disease or condition related to orexin receptor 1 or orexin receptor 2 is selected from the group consisting of obesity, migraine, cluster headache, narcolepsy, Parkinson's disease, Alzheimer's disease, depression, addictions, anxiety, cancer, diabetes, insomnia, irritable bowel syndrome, neuropathic pain, pain, schizophrenia, sleep disorder, and Tourette syndrome. In other embodiments, the disease or condition related to somatostatin receptor 2 is selected from the group consisting of Crushing's syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain and pain. In still some embodiments, the disease or condition related to dopamine D_{2L} receptor is selected from the group consisting of schizophrenia, anxiety, depression, migraine, pain, Parkinson's disease, addiction and Tourette syndrome.

It is the third objective of this disclosure to provide a pharmaceutical composition for treating a disease or condition related to orexin receptor 1, orexin receptor 2, somatostatin receptor 2 or dopamine D_{2L} receptor in a subject, comprising verapamil and a pharmaceutically acceptable excipient, wherein the verapamil is any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof. In one preferred embodiment, the verapamil is (R)-(+)-verapamil, a pharmaceutically acceptable derivative, stereoisomer, metabolite, salt or solvate thereof, or a combination thereof.

The details of one or more embodiments of the invention are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### DISCLOSURE OF INVENTION

The practices of this invention are hereinafter described in detail with respect to methods and compositions for treating a disease or condition related to orexin receptor 1, orexin receptor 2, somatostatin receptor 2 or dopamine D_{2L} receptor in a subject.

The inventors of this disclosure unexpectedly identify that verapamil is capable of binding to orexin receptor 1, orexin receptor 2, somatostatin receptor 2 or dopamine D_{2L} receptor. Specifically, verapamil act as an agonist to somatostatin receptor 2; and an antagonist to orexin receptor 1, orexin receptor 2 or dopamine D_{2L} receptor. Accordingly, one aspect of this disclosure is directed to a method of binding at least one receptor selected from the group consisting of orexin receptor 1, orexin receptor 2, somatostatin receptor 2 or dopamine D_{2L} receptor in a subject, comprising administering verapamil to the subject.

The term “verapamil (2-(3,4-dimethoxyphenyl)-5-{[2-(3,4-dimethoxyphenyl)ethyl]-(methyl)amino}-2-prop-2-ylpentanenitrile)” herein refers to verapamil, its stereoisomers, derivatives, metabolites, salts, solvates and/or combinations thereof, and may be any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof. Verapamil is known to exist in either (R)- or (S)-form. It is well known that pharmacodynamics and pharmacokinetics of the (R)-(+)-verapamil and (S)-(−)-verapamil differs in vivo, with (S)-form more potent than the (R)-form. In one embodiment of this disclosure, verapamil is provided in (R)-form. In another example, verapamil is provided in (S)-form. In some examples, verapamil is proved as a mixture of (R)-(+)-verapamil and (S)-(−)-verapamil, for example, about 90% (R)-form and about 10% (S)-form, about 80% (R)-form and about 20% (S)-form, about 70% (R)-form and about 30% (S)-form, about 60% (R)-form and about 40% (S)-form, about 40% (R)-form and about 60% (S)-form, about 30% (R)-form and about 70% (S)-form, about 20% (R)-form and about 80% (S)-form, or about 10% (R)-form and about 90% (S)-form. In one specific example, verapamil exists as a racemic mixture containing approximately equal amount of (R)-(+)-verapamil and (S)-(−)-verapamil, that is, with (R)- and (S)-form being about 50%, respectively.

The term “a salt” refers herein a salt which is formed by the interaction of a base (such as verapamil in this disclosure) with an acid, including organic or inorganic types of acids such as hydrochloric acid, hydrobromic acid, hydroiodic acid, perchloric acid, sulfuric acid, nitric acid, phosphoric acid, propionic acid, glycolic acid, acetic acid, pyruvic acid, malonic acid, succinic acid, maleic acid, fumaric acid, tartaric acid, citric acid, benzoic acid, carbonic acid, cinnamic acid, methanesulfonic acid, ethanesulfonic acid, hydroxyethanesulfonic acid, benzenesulfonic acid, p-toluene sulfonic acid, cyclohexanesulfamic acid, salicyclic acid, p-aminosalicyclic acid, 2-phenoxybenzoic acid and 2-acetoxybenzoic acid. In one preferred example, the salt is verapamil hydrochloride. The term “solvate” herein refers to a complex formed by the interaction of a compound (such as verapamil in this disclosure) with surrounding solvent molecules, such as water, ethanol, and etc. In one example, the solvate is verapamil hydrochloride hydrate.

The term “racemic” as used herein refers to a mixture of the enantiomers, or stereoisomers, of a pharmaceutically active agent, in which neither enantiomer or stereoisomer is substantially purified from the other.

In another aspect of the present disclosure, it is directed to a method of treating a disease or condition related to orexin receptor 1, orexin receptor 2, somatostatin receptor 2 or dopamine D_{2L} receptor, comprising administering to a subject an effective amount of verapamil and a pharmaceutically acceptable excipient.

The term “treating” as used herein may involve prevention or prophylaxis of a disease, condition or other adverse physiological event in a susceptible individual as well as treatment of a clinically symptomatic individual by ameliorating, mitigating or alleviating the symptoms of the disease, condition or other adverse physiological event.

As described above, verapamil may be any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof.

The term “pharmaceutically acceptable excipient” refers to compounds that are compatible with other ingredients in a pharmaceutical composition and would not endanger the subject after administering.

The term “agonist” refers herein agents or drugs that upon binding to their receptors elicit or trigger a biological response.

The term “antagonist” refers herein agents or drugs that neutralize or impede the action or effects of others, e.g., a drug that binds to a receptor without eliciting a biological response and effectively blocking the binding of a substance that could elicit such a response.

The disease or condition related to orexin receptor 1 or 2 is selected from the group consisting of obesity, migraine, cluster headache, Parkinson's disease, Alzheimer's disease, depression, addictions, anxiety, cancer, diabetes, insomnia, irritable bowel syndrome, narcolepsy, neuropathic pain, pain, schizophrenia, sleep disorder, and Tourette syndrome. In other embodiments, the disease or condition related to somatostatin 2 receptor is selected from the group consisting of Crushing's syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain and pain. In still some embodiments, the disease or condition related to dopamine D_{2L} receptor is selected from the group consisting of schizophrenia, anxiety, depression, migraine, pain, Parkinson's disease, Tourette syndrome and addiction.

Thus, it is a further aspect of this disclosure to provide a pharmaceutical composition for treating a disease or condition related to orexin receptor 1, orexin receptor 2, somatostatin receptor 2 or dopamine D_{2L} receptor in a subject, comprising a therapeutically effective amount of verapamil and a pharmaceutically acceptable excipient. The verapamil, the pharmaceutically acceptable excipient and the disease or condition related to any of the receptor are as described above.

The pharmaceutical composition of this disclosure may be administered by oral, parenteral (e.g., intramuscular, intraperitoneal, intravenous or subcutaneous injection or implant), nasal, sublingual, topical or transdermal routes and can be formulated into various dosage forms suitable for each route of administration. A number of suitable dosage forms are described below, but are not meant to include all possible choices. One of skilled person in the art is familiar with the various dosage forms that are suitable for use in each route. It is to be noted that the most suitable route in any given case would depend on the nature or severity of the disease or condition being treated. The active ingredient, such as verapamil, is mixed with at least one pharmaceutically acceptable excipient including, but are not limited to, fillers, binders, lubricants, glidants, disintegrants and the like. Suitable fillers may include one or more of lactose, microcrystalline cellulose, polyethylene glycols, mannitol, calcium phosphate, calcium sulfate, kaolin, dry starch, sorbitol, powdered sugar, prosolv, and the like. Suitable disintegrants may include one or more of starch, croscarmellose sodium, crospovidone, sodium starch glycolate, hydroxypropylcellulose, and the like. Suitable glidants may include one or more of colloidal silicon dioxide, talc or cornstarch, and the like. Suitable lubricants comprises one or more of hydrogenated vegetable oil, hydrogenated castor oil, light mineral oil, glycerol monostearate, glycerol monobehenate, glyceryl behenate, glyceryl palmitostearate, and the like.

In some embodiments, the pharmaceutical compositions of this disclosure are solid dosage forms for oral administration. Such solid dosage forms may be capsules, sachets, tablets, pills, lozenges, powders or granules. In such forms, the active ingredient such as verapamil is mixed with at least one pharmaceutically acceptable excipient as described above. Any of the described solid dosage forms may optionally contain coatings and shells, such as enteric coatings, and coatings for modifying the release rate of any of the ingredients. Examples of such coatings are well known in the art. In one example, the pharmaceutical compositions of this disclosure are tablets such as quick-release tablets. In still another example, the pharmaceutical compositions of this disclosure are formulated into sustained release forms. In another example, the pharmaceutical compositions of this disclosure are powders that are encapsulated in soft and hard gelatin capsules.

In some embodiments, the pharmaceutical compositions of this disclosure are liquid dosage forms for oral administration. The liquid formulation may further include a buffering agent to maintain a desired pH. The liquid dosage formulations may also be filled into soft gelatin capsules. For example, the liquid may include a solution, suspension, emulsion, microemulsion, precipitate or any desired liquid media carrying (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof. The liquid may be designed to improve the solubility of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof to form a drug-containing emulsion or to disperse phase upon release. In such forms, the active ingredient is mixed with at least one pharmaceutically acceptable excipient as described above.

In some embodiments, the pharmaceutical compositions of this disclosure are formulations suitable for parenteral administration, such as administration by injection, which includes, but is not limited to, subcutaneous, bolus injection, intramuscular, is intraperitoneal and intravenous injection. The pharmaceutical compositions may be formulated as isotonic suspensions, solutions or emulsions in oily or aqueous vehicles, and may contain formulatory agents such as suspending, stabilizing or dispersing agents. Alternatively, the compositions may be provided in dry form such as powders, crystallines or freeze-dried solids with sterile pyrogen-free water or isotonic saline before use. They may be presented in sterile ampoules or vials. In such forms, the active ingredient, such as verapamil, is mixed with at least one pharmaceutically acceptable excipient as described above.

In some embodiments, the pharmaceutical compositions of this disclosure are formulations suitable for intranasal administration or administration by inhalation. The compositions are delivered in the form of a solution or suspension from a pump spray container that is squeezed or pumped by the patient or in the form of a dry powder or as an aerosol spray from a pressurized container or a nebulizer, with the use of a suitable propellant such as dichlorodifluoromethane, trichlorofluoromethane, dichlorotetrafluoroethane, a hydrofluoroalkane such as 1,1,1,2-tetrafluoroethane (HFA 134A™) or 1112333-heptafluoropropane (HFA 227EA™), carbon dioxide or other suitable gas. In the case of a pressurized aerosol, the dosage unit may be determined by providing a valve to deliver a metered amount. The pressurized container or nebulizer may contain a solution or suspension of the composition of this disclosure. Capsules or cartridges for use in an inhaler or insufflators may be formulated to contain a powder mix of the active compound of this disclosure such as (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof. In such forms, the active ingredient is mixed with at least one pharmaceutically acceptable excipient as described above.

In some embodiments, the pharmaceutical compositions of this disclosure are dosage formulations that are suitable for topical or transdermal administration. Such formulations include sprays, ointments, pastes, creams, lotions, gels, solutions and patches. Such formulations may optionally include excipients such as animal and vegetable fats, oils, waxes, paraffins, starch, tragacanth, cellulose derivatives, silicons, bentonites, silicic acid, talc, zinc oxide or mixtures thereof. Sprays may also include excipients such as talc, silicic acid, aluminum hydroxide and calcium silicate; additionally, sprays may contain propellants such as chlorofluoro-hydrocarbons and volatile hydrocarbons such as butane and propane. Transdermal patches may be made by dissolving, dispersing or incorporating a pharmaceutical composition of the present disclosure in a suitable medium, such as elastomeric matrix material. Absorption enhancers may also be used to increase the flux of the mixture across the skin. Alternatively, the composition of the present disclosure may be formulated into a lotion or a cream. In such forms, the active ingredient, such as verapamil is mixed with at least one pharmaceutically acceptable excipient as described above.

It must be noted that as used herein and in the appended claims, the singular forms “a”, “an”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes.

The following Examples are provided to illustrate certain aspects of the present invention and to aid those of skilled in the art in practicing this invention. These Examples are in no way to be considered to limit the scope of the invention in any manner.

### EXAMPLES

### Binding Affinity Assay

(R)-(+)-verapamil hydrochloride and (S)-(−)-verapamil hydrochloride were obtained from Sigma-Aldrich® (USA). Binding activity was evaluated to assess the selectivity of verapamil on various receptors. The binding activity of the reported receptors was analyzed using the following known methods. Primary screening were performed in duplicate and presented in semi-quantitative data (e.g., IC₅₀, Kᵢ and n_{H}). Where presented, IC₅₀ values were determined by a non-linear, least squares regression analysis using MathIQ™ (ID Bussiness Solutions Ltd., UK). Where inhibition constants (K) presented, the Kᵢ value were calculated using the equation of Cheng and Prusoff (Cheng. Y., Prusoff, W. H., (1973) Biochem. Pharmacol. 22:3099-3108) using the observed IC₅₀ of the tested compound, the concentration of radioligand employed in the assay and the historical values of the K_{D} of the ligand. Where presented, the Hill coefficient (n_{H}), defining the slope of the competitive binding curve, was calculated using MathIQ™.

Orexin receptor 1 or 2 binding affinity was examined using the methods disclosed by Kukkonen et al., (2002) Am J Physiol Cell Physiol. 283:C1567-C1591; and Langmead et al., (2004) Br J Pharmacol. 141(2):340-346. Calcium channel (L-type) binding activity was examined using the methods disclosed by Church and Zsotér, (1980) Can J Physiol. Pharmacol. 58:254-264; and Spedding. (1984) M. Eur J Pharmacol. 83:211. Somaostatin receptor 2 binding activity was estimated by the method described by Feniuk et al., (1993) Br J Pharmacol. 110:1156-1164. Adenosine receptor binding activity was examined using the method described by Varani et al., (1996) Br J Pharmacol. 117:1693-1701. Bradykinin receptor binding activity was examined using the methods described by Horlick et al., (1999) Immunopharmacology 43(2-3):169-177; and Phagoo et al., (2001) J Pharmacol Exp Ther 298(1):77-85. Calcium channel (N-type) binding activity was examined using the method disclosed by Moresco et al., (1990) Neurobiol of Aging. 11(4):433-436. Cannabinoid receptor binding activity was examined using the method disclosed by Reggio et al., (1998) J Med Chem 41(26):5177-5187. Corticotrophin releasing factor (CRF) receptor binding activity was examined using the methods disclosed by Lewis et al., (2001) Proc Natl Acad Sci USA 98(13):7570-7575; and Sutton et al., (1995) Endocrinology 136(3):1097-1102. Estrogen receptors (including ERα and ERβ) were examined using the method disclosed by Obourn et al., (1993) Biochem 32:6229-6236. GABA receptor (including GABA_{A} and GABA_{B1A},) binding activity was examined using methods disclosed by Lewin et al., (1989) Mol Pharmacol 35:189-194; Maksay G. (1993) Eur J Pharmacol 246:255-260; Enna and Snyder, (1977) Mol Pharmacol. 13:442-453; Martini et al., (1983) J Neurochem. 41:1183-1185; Damm et al., (1978) Res Comm Chem Pathol Pharmacol. 22:597-600; Speth et al., (1979) Life Sci. 24:351-357; Dubinsky et al., (2002) J Pharmacol Exp Ther. 303(2):777-790; and McLeod et al., (2002) Brain Res Mol Brain Res 104(2):203-209. Leptin receptor binding activity was examined using the method disclosed by Fong et al., (1998) Mol Pharmacol 53:234-240. Nicotinin acetylcholine receptor (including (α1, α4β2 and α7) binding activity was examined using the methods disclosed by Davila-Garcia et al., (1997) J Pharmacol Exp Ther 282:445-451 ; and Whiteaker et al., (2000) Br J Pharmacol. 131:729-739. Sodium channel binding activity was examined using the method disclosed by Catterall et al., (1981) J Biol Chem. 256:8922-8927. Dopamine receptor binding activity was examined using the methods disclosed by Lewis et al., (2001) Proc Natl Acad Sci USA 98(13):7570-7575; and Sutton et al., (1995) Endocrinology 136(3):1097-1102. Glutamate receptor binding activity was examined using the method disclosed by Mutel et al., (2000) J Neurochem. 75(6):2590-2601. Tachykinin receptor binding activity was examined using the method disclosed by Patacchini and Maggi, (1995) Arch Int Pharmacodyn. 329:161-184. Vanilloid receptor binding activity was examined using the methods disclosed by Szallasi et al., (1993) J Pharmacol Exp Ther. 267(2):728-733. Melatonin receptor binding activity was examined using the methods disclosed by Beresford et al., (1998) J Pharmacol Exp Ther 285(3):1239-1245; and Paul et al., (1999) J Pharmacol Exp Ther 290(1):334-340. Serotonin receptor binding activity was examined using the methods disclosed by Bonhaus et al., (1995) Br J Pharmacol 115:622-628; and Saucier and Albert (1997) J. Neuorchem 68:1998-2011.

Binding on various receptors as described above were tested, and significant binding of (R)-(+)-verapamil hydrochloride and (S)-(−)-verapamil hydrochloride were observed in orexin receptor 1, orexin receptor 2, somatostatin receptor 2, sodium channel, L-type and N-type calcium channels, serotonin, melatonin and dopamine (D_{2L}) receptors (some data were not shown). Among them, binding of (R)-(+)-verapamil hydrochloride and (S)-(−)-verapamil hydrochloride with orexin receptor 1, orexin receptor 2, somatostatin receptor 2 are novel discovery of this application and have never been reported before.

Table 1 is a summary of the binding affinity results of (R)-(+)-verapamil hydrochloride and (S)-(−)-verapamil hydrochloride on orexin receptor 1, orexin receptor 2 and sodium channel. In the orexin assay, (R)-(+)-verapamil hydrochloride showed significant activity on orexin receptor 1 with an IC₅₀ value of 242 μM (Kᵢ=170 μM and n _{H}=1.31), and orexin receptor 2 with an IC₅₀ value of 83.7 μM (Kᵢ=64 μM and n_{H}=1.81). (S)-(−)-verapamil hydrochloride also showed significant activity on orexin receptor 1 with an IC₅₀ value of 69.8 μM (Kᵢ=49 μM and n_{H}=1.27), and orexin receptor 2 with an IC₅₀ value of 34.7 μM (Kᵢ=26.5 μM and n_{H}=1.33). For sodium channel, both (R)-(+)-verapamil hydrochloride and (S)-(−)-verapamil hydrochloride showed significant binding activity with an IC₅₀ value of 3.28 μM and 1.11 μM, respectively. Among them, binding of (R)-(+)-verapamil hydrochloride and (S)-(−)-verapamil hydrochloride with orexin receptor 1, orexin receptor 2 are novel discovery of this application and have never been reported before.

Further screening studies using SelectScreen® Cell-based GPCR Profiling Service provided by Invitrogen (Madison, Wis., USA) confirmed that both (R)-(+)-verapamil hydrochloride and (S)-(−)-verapamil hydrochloride act as antagonists to orexin receptor 1 and orexin receptor 2. Both agonist and antagonist assays were performed in accordance with the manufacturer's protocols, and (R)-(+)-verapamil hydrochloride and (S)-(−)-verapamil hydrochloride used in this assay were obtained from Syn-Tech Chem & Pharm Co. Ltd (Taiwan, R.O.C.). Results are provided in Table 2.

Table 3 summarizes the binding activity of (R)-(+)-verapamil hydrochloride and (S)-(−)-verapamil hydrochloride on L-type calcium channel (including atrial inotropy and ileum), somatostatin receptor 2 and dopamine (D_{2L}) receptor. In the somatostatin tissue assay, both (R)-(+)-verapamil hydrochloride and (S)-(−)-verapamil hydrochloride showed significant agonist activity with EC₅₀ value of 1.6 μM and 0.19 μM, respectively. In the calcium channel L-type, atrial inotropy tissue assay, both (R)-(+)-verapamil hydrochloride and (S)-(−)-verapamil hydrochloride showed significant antagonist activity with IC₅₀ value of 21.9 μM and 6.2 μM, respectively. In the calcium channel L-type, Ileum tissue assay, both (R)-(+)-verapamil hydrochloride and (S)-(−)-verapamil hydrochloride showed significant antagonist activity with IC₅₀ value of 0.04 μM and 6.8 nM, respectively. In the dopamine (D_{2L}) receptor assay, both (R)-(+)-verapamil hydrochloride and (S)-(−)-verapamil hydrochloride showed significant antagonist activity with IC₅₀ value of 5.97 μM and 1.47 μM, respectively. Among them, the agonist activities of (R)-(+)-verapamil hydrochloride and (S)-(−)-verapamil hydrochloride of somatostatin receptor 2, and the antagonist activities of (R)-(+)-verapamil hydrochloride and (S)-(−)-verapamil hydrochloride of dopamine (D_{2L}) receptor are novel discovery of this application and have never been reported before.

TABLE 1
Binding Activity of (R)-(+)-verapamil hydrochloride and (S)-(−)-verapamil
hydrochloride on orexin receptor 1 or 2 and Sodium Channel

| Receptor/ | | Conc. | % | IC₅₀ | Kᵢ | |
|---|---|---|---|---|---|---|
| Compound | Species | (μM) | Inhibition | (μM) | (μM) | n_{H} |
| Orexin receptor 1 | | | | | | |
| (R)-(+)-verapamil | Hum | 300 | 57 | 242 | 170 | 1.31 |
| hydrochloride | | | | | | |
| (S)-(−)-verapamil | hum | 100 | 58 | 69.8 | 49 | 1.27 |
| hydrochloride | | | | | | |
| Orexin receptor 2 | | | | | | |
| (R)-(+)-verapamil | hum | 100 | 56 | 83.7 | 64 | 1.81 |
| hydrochloride | | | | | | |
| (S)-(−)-verapamil | hum | 100 | 79 | 34.7 | 26.5 | 1.33 |
| hydrochloride | | | | | | |
| Sodium Channel, | | | | | | |
| Site 2 | | | | | | |
| (R)-(+)-verapamil | rat | 10 | 72 | 3.28 | 2.99 | 0.938 |
| hydrochloride | | | | | | |
| (S)-(−)-verapamil | rat | 3 | 75 | 1.11 | 1.02 | 1.1 |
| hydrochloride | | | | | | |

Hum = human

TABLE 2
Agonist/Antagonist Assay Results of (R)-(+)-verapamil
hydrochloride and (S)-(−)-verapamil hydrochloride on
orexin receptor 1 or 2

| | Agonist | Antagonist |
|---|---|---|
| Receptor/Compound | EC₅₀ (μM) | IC₅₀ (μM) |
| Orexin receptor 1 | | |
| (R)-(+)-verapamil | >300 | 136 |
| hydrochloride | | |
| (S)-(−)-verapamil | >300 | 104 |
| hydrochloride | | |
| Orexin receptor 2 | | |
| (R)-(+)-verapamil | >300 | 198 |
| hydrochloride | | |
| (S)-(−)-verapamil | >300 | 176 |
| hydrochloride | | |

TABLE 3
Binding Activity of (R)-(+)-verapamil hydrochloride and
(S)-(−)-verapamil hydrochloride on L-type calcium channel,
somatostatin receptor 2 and Dopamine D_{2L} receptor

| | | | | AG. | ANT. | | |
|---|---|---|---|---|---|---|---|
| Receptor/Compound | Species | Conc. | | (%) | (%) | EC₅₀/IC₅₀ | |
| L Type Calcium Channel, | | | | | | | |
| Atrial Inotropy (Left artria) | | | | | | | |
| (R)-(+)-verapamil | gp | 30 | μM | 0 | 61 | 21.9 | μM |
| hydrochloride | | | | | | | |
| (S)-(−)-verapamil | gp | 10 | μM | 0 | 61 | 6.2 | μM |
| hydrochloride | | | | | | | |
| L Type Calcium Channel, | | | | | | | |
| Ileum | | | | | | | |
| (R)-(+)-verapamil | gp | 0.1 | μM | 0 | 83 | 0.04 | μM |
| hydrochloride | | | | | | | |
| (S)-(−)-verapamil | gp | 10 | nM | 0. | 66 | 6.8 | nM |
| hydrochloride | | | | | | | |
| Somatostatin receptor 2 | | | | | | | |
| (R)-(+)-verapamil | gp | 3 | μM | 69 | ND | 1.6 | μM |
| hydrochloride | | | | | | | |
| (S)-(−)-verapamil | gp | 0.3 | μM | 61 | ND | 0.19 | μM |
| hydrochloride | | | | | | | |
| Dopamine D_{2L} | | | | | | | |
| (R)-(+)-verapamil | hum | 10 | μM | ND | 58 | 5.97 | μM |
| hydrochloride | | | | | | | |
| (S)-(−)-verapamil | hum | 3 | μM | ND | 66 | 1.47 | μM |
| hydrochloride | | | | | | | |

Hum = human,
gp = guinea pig
AG. = Agonist;
ANT. = Antagonist;
ND = not detected

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A use of verapamil for the manufacture of a medicament for treating a disease or condition related to orexin receptor 1, orexin receptor 2, somatostatin receptor 2 or dopamine D_{2L}, receptor, wherein the verapamil is capable of binding to any of the orexin receptor 1, orexin receptor 2, somatostatin receptor 2 or dopamine D_{2L}, receptor.

2. The use of claim 1, wherein the verapamil is any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof.

3. The use of claim 2, wherein the verapamil is any of (R)-(+)-verapamil, a pharmaceutically acceptable derivative, stereoisomer, metabolite, salt, or solvate thereof or a combination thereof.

4. The use of claim 1, wherein the disease or condition related to orexin receptor 1 or orexin receptor 2 is selected from the group consisting of obesity, migraine, cluster headache, Parkinson's disease, Alzheimer's disease, depression, addictions, anxiety, cancer, diabetes, insomnia, irritable bowel syndrome, narcolepsy, neuropathic pain, pain, schizophrenia, sleep disorder, and Tourette syndrome.

5. The use of claim 1, wherein the disease or condition relates to somatostatin receptor 2 is selected from the group consisting of Crushings syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain and pain.

6. The use of claim 1, wherein the disease or condition related to dopamine D_{2L} receptor is selected from the group consisting of schizophrenia, anxiety, depression, migraine, pain, Parkinson's disease, Tourette syndrome and addiction.

7. A method of in vitro binding of verapamil with at least one of orexin receptor 1, orexin receptor 2, somatostatin receptor 2 or dopamine D_{2L} receptor in a cell, comprising the step of administering an effective amount of verapamil to the cell, wherein the verapamil is any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof.

8. The method of claim 7, wherein the verapamil is (R)-(+)-verapamil, a pharmaceutically acceptable derivative, stereoisomer, metabolite, salt, or solvate thereof or a combination thereof.

9. A method of treating a subject having a disease or condition related to orexin receptor 1, orexin receptor 2, somatostatin receptor 2 or dopamine D_{2L} receptor, comprising administering an effective amount of verapamil to the subject.

10. The method of claim 9, wherein the verapamil is any of (R)-(+)-verapamil, (S)-(−)-verapamil, a mixture of (R)-(+)-verapamil and (S)-(−)-verapamil, a pharmaceutically acceptable derivative, stereoisomer, metabolite, salt or solvate thereof, or a combination thereof.

11. The method of claim 10, wherein the verapamil is (R)-(+)-verapamil, a pharmaceutically acceptable derivative, stereoisomer, metabolite, salt, or solvate thereof or a combination thereof.

12. The method of claim 9, wherein the disease or condition related to orexin receptor 1 or orexin receptor 2 is selected from the group consisting of obesity, migraine, cluster headache, Parkinson's disease, Alzheimer's disease, depression, addictions, anxiety, cancer, diabetes, insomnia, irritable bowel syndrome, narcolepsy, neuropathic pain, pain, schizophrenia, sleep disorder, and Tourette syndrome.

13. The method of claim 9, wherein the disease or condition relates to somatostatin receptor 2 is selected from the group consisting of Crushings syndrome, gonadotropinoma, gastrinoma, Zollinger-Ellison syndrome, hypersecretory diarrhea related to AIDS and other conditions, irritable bowel syndrome, pancreatitis, Crohn's disease, systemic sclerosis, thyroid cancer, psoriasis, hypotension, panic attacks, scleroderma, small bowel obstruction, gastroesophageal reflux, duodenogastric reflux, Grave's disease, polycystic ovary disease, upper gastrointestinal bleeding, pancreatic pseudocyst, pancreatic ascites, leukemia, meningioma, cancer cachexia, acromegaly, restenosis, hepatoma, lung cancer, melanoma, wasting, type 2 diabetes, Syndrome X, fibrosis, hyperlipidemia, hyperamylinemia, hyperprolactinemia, prolactinomas, cluster headache, depression, neuropathic pain and pain.

14. The method of claim 9, wherein the disease or condition related to dopamine D_{2L} receptor is selected from the group consisting of schizophrenia, anxiety, depression, migraine, pain, Parkinson's disease, Tourette syndrome and addiction.

15. Use of verapamil as an agonist of somatostatin receptor 2.

16. Use of verapamil as an antagonist of orexin receptor 1, orexin receptor 2 or dopamine D_{2L} receptor.
